(19) United States
(12) Patent Application Publication
(10) Pub. No.: US 2024/0009371 A1
(43) Pub. Date: Jan. 11, 2024

(54) PUMPING CASSETTE FOR TISSUE TREATMENT DEVICES

(71) Applicant: Trice Medical, Inc., Malvern, PA (US)

(72) Inventors: Angelo Francis Patrinicola, Malvern, PA (US); Kyle Joseph VanLeer, Eagleville, PA (US); Sam de la Torre, Malvern, PA (US); Bruce J. Richie, Malvern, PA (US); Larry A. Woods, Malvern, PA (US); Virginia W. Njagi, Malvern, PA (US)

(21) Appl. No.: 18/347,454

(22) Filed: Jul. 5, 2023

Related U.S. Application Data
(60) Provisional application No. 63/367,805, filed on Jul. 6, 2022.

(51) Int. Cl. A61M 1/00

(52) U.S. Cl. CPC A61M 1/774; A61M 1/72; A61M 1/784; A61M 2205/3334

(57) ABSTRACT

Methods, apparatuses, and systems for cutting anatomic members with minimally invasive endoscopic ultrasound are described. A disposable cutting handpiece and cassette can be attached to a reusable main console for the procedure and then separated to be discarded and/or analyzed. The handpiece may include a power line, aspiration, and/or irrigation. The cassette may contain one or more pumps and an electronic connection that interfaces with a console deliver power to the handpiece, and optionally to identify characteristics such as probe and/or handpiece type. The cassette may further include sensors, a sensor interface, and/or a flow correction circuit for reducing unwanted flow blockages or vacuum.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### BACKGROUND

### Field of the Invention

The disclosed device concerns surgical instruments employed in minimally invasive surgery. More particularly, the disclosed device and method relate to an apparatus and method employable as a disposable ultrasonic cutting instrument that is capable of irrigation and aspiration by means of an integrated pump.

This disclosure pertains to methods, apparatus, and systems for performing, for example, endoscopic treatment of chronic tendinosis or fasciitis via ultrasonic cutting, as well as similar surgical procedures on other suitable tissues.

### Description of the Related Art

Repetitive motion or use of body tissues may cause injuries or painful conditions to arise. For example, tennis elbow, or lateral epicondylalgia is a clinical syndrome in which patients experience pain at the lateral elbow. Such pain in the lateral elbow may worsen over time and, despite adequate treatment, many patients develop chronic symptoms and eventually become candidates for surgical treatment.

A number of surgical procedures have been described to treat chronic tendinosis or fasciitis affecting various regions of the body. Particular open techniques typically require open surgical dissection down to the pathological tissue and therefore necessitate repair of the surgically compromised normal tissue. Some arthroscopic techniques may be slightly less invasive, but several such procedures have been associated with neurological complications and may require the use of a high-cost operating suite and associated personnel. Various percutaneous techniques have been described which release, ablate, or resect the pathological tissue. These percutaneous techniques, however, generally require a noticeable skin incision, some surgical dissection, and the aforementioned use of a high-cost operating suite and supportive equipment and personnel.

Current devices known in the art suffer from deficiencies such as insufficient aspiration and/or an unbalanced irrigation to aspiration ratio. As such, there is a continuing unmet need for an improved disposable surgical cassette with pumps that interface with a pump motor and power source to supply power, aspiration and irrigation to a distal surgical ultrasound delivery handle. Such a device should provide sensing capabilities, including pressure, bubble, connection detection, and identification as well as flow correction capabilities in the event of blockage or vacuum.

### SUMMARY

The embodiments disclosed herein each have several aspects directed toward soft and/or hard tissue treatment. Without limiting the scope of this disclosure, multiple embodiments and/or examples will now be briefly discussed. After considering this discussion, and particularly after reading the section entitled “Detailed Description,” one of skill in the art will understand how the features of the embodiments described herein provide advantages over existing systems, devices and methods.

Disclosed herein are methods, apparatuses, and systems for cutting anatomic members, such as tendons and fascia, with endoscopic ultrasound. The apparatus may be a disposable cutting handpiece and cassette that are attached to a reusable main console for the procedure, and then safely separated to be discarded and/or analyzed. The apparatus may also include the ability to store material, for example, material collected from the aspirating handpiece for disposal or analysis. The cutting handpiece may use piezoelectric crystals to create ultrasonic cutting action, and may optionally include a beveled tip, an aspiration conduit, and/or an irrigation conduit. The cassette may contain one or more diaphragm pumps, for example, one pump for irrigation and one for aspiration, operated by one or more motors housed in the console. The cassette may include sensors, for example to detect pressure or flow, presence of bubbles, proper connection, and system or characteristic identification. The cassette may include an interface for cooperating with one or more sensors in the console. The cassette may include an electronic connection that interfaces with a console deliver power to the handpiece, and optionally to identify characteristics such as probe and/or handpiece type and performance characteristics. The cassette may further include a flow correction circuit for reducing unwanted flow blockages or vacuum.

In some aspects, the techniques described herein relate to a fluid irrigation and/or aspiration apparatus including: a reusable portion with a pump motor and a power source; and a disposable portion including: a handpiece; a power line connected to the handpiece and connectable to the power source; and a diaphragm. When the reusable portion is connected to the disposable portion at a connection, the power source interfaces with the power line to deliver power to the handpiece, the pump motor interfaces with the diaphragm to create a diaphragm pump configured to provide a flow of a fluid in the disposable portion; and the disposable portion separates the fluid from the reusable portion. In some aspects, the disposable portion also includes handpiece identification electronics and/or valves.

In some aspects, the techniques described herein relate to an apparatus, further wherein the fluid is an irrigation fluid, the apparatus further including a source of the irrigation fluid, and wherein the diaphragm pump provides the irrigation fluid to the handpiece. In some aspects, the apparatus further includes a collector and the diaphragm pump provides flow of aspirated fluid from the handpiece to the collector. In some aspects, the apparatus includes a tube to carry the flow of the fluid. In some aspects, the tube includes a filter. In some aspects, the filter is located to protect the diaphragm pump from particles in the flow of the fluid.

In some aspects, the apparatus connection includes a coupling mechanism that transmits motion from the diaphragm pump to the diaphragm. In some aspects, the connection includes one or more sensing devices to sense flow, bubbles, and/or pressure. In some aspects, the disposable portion includes one or more sensing devices to sense flow, bubbles, and/or pressure. In some aspects, the apparatus includes a collector wherein the flow is an aspiration from the handpiece directed to the collector. In some aspects, the collector is a removable collection bag.

In some aspects, the techniques described herein relate to a disposable fluid irrigation and aspiration apparatus including: a power line configured to connect to a handpiece and connectable to a power source in a reusable console; handpiece identification electronics; an irrigation diaphragm; an aspiration diaphragm; valves; and a connection area configured to connect to the reusable console. When the connection area is connected to the reusable console, the power line receives power to be delivered to a handpiece, the irrigation diaphragm interfaces with a pump motor in the reusable console to provide an irrigation flow, the aspiration diaphragm interfaces with a pump motor in the reusable console to provide an aspiration flow, and the irrigation flow and the aspiration flow are separated from the reusable console. In some aspects, the apparatus includes a filter. In some aspects, the apparatus includes the handpiece. In some aspects, the handpiece includes identification electronics, and software reads the handpiece identification and loads the performance parameters. In some aspects, the apparatus includes one or more sensing devices to sense flow, bubbles, and/or pressure. In some aspects, the apparatus includes a collector wherein the aspiration flow directs fluid from the handpiece to the collector. In some aspects, the collector is a removable collection bag.

### BRIEF DESCRIPTION OF THE DRAWINGS

The foregoing and other features, aspects, and advantages of the embodiments of the systems, apparatuses, and methods described herein are described in detail below with reference to the drawings of various embodiments, which are intended to illustrate and not to limit the embodiments of the invention. The drawings comprise the following figures in which:

FIG. 1 is a perspective view of one example embodiment of the system disclosed herein.

FIG. 2 is a schematic view of one example of the controller, illustrating the command module, the use interface, and the tubing cassette.

FIGS. 3A, 3B and 3C are alternative views of one example cassette.

FIG. 4 illustrates an example of an ultrasonic handpiece.

FIG. 5 illustrates an example of an ultrasonic handpiece with electrical and tubing connections.

FIGS. 6A and 6B illustrate an example of an ultrasonic handpiece with electrical and tubing connections connected to a cassette and collection bag.

FIG. 7 illustrates an example of a cassette and console.

FIG. 8 illustrates an implementation of electrical and tubing connections inside a cassette.

FIG. 9 is an exploded view of an implementation of a cassette.

FIG. 10 is a perspective view of an implementation of a cassette showing the front of the cassette.

FIG. 11 is a perspective view of an implementation of a cassette showing the back of the cassette.

FIG. 12 illustrates an implementation of a cassette and console with sensors.

FIG. 13A illustrates an example of the connection area of a console.

FIG. 13B illustrates an example of the connection area of FIG. 13A with a cassette partially inserted.

FIG. 13C illustrates an example of the connection area of FIG. 13A with a cassette fully inserted.

FIG. 14 illustrates an example of the connection area of FIG. 13A from the inside of the console.

FIG. 15 illustrates an implementation of fluid flow paths within a cassette.

FIG. 16A illustrates an implementation of fluid flow paths within a cassette while a solenoid valve is shut.

FIG. 16B illustrates an implementation of fluid flow paths within a cassette while the solenoid valve of FIG. 16A is open.

Throughout the drawings, unless otherwise noted, reference numbers may be re-used to indicate a general correspondence between referenced elements. The drawings are provided to illustrate example embodiments described herein and are not intended to limit the scope of the disclosure.

### DETAILED DESCRIPTION

Disclosed herein are methods, apparatuses, and systems for cutting anatomic members, such as tendons, diseased bone, foot ulcers, and fascia, with endoscopic ultrasound. The apparatus may be a disposable cutting handpiece and cassette that are attached to a main console for the procedure and then safely separated to be discarded and/or analyzed. The apparatus may also include the ability to store material, for example, material collected from the aspirating handpiece for disposal or analysis. The cutting handpiece may use piezoelectric crystals to create ultrasonic cutting action, and may optionally include a beveled tip, an aspiration conduit, and/or an irrigation conduit. The cassette may contain one or more diaphragm pumps, for example, one pump for irrigation and one for aspiration, operated by one or more motors housed in the console. The cassette may include sensors, for example to detect pressure or flow, presence of bubbles, proper connection, and system or characteristic identification. The cassette may include an interface for cooperating with one or more sensors in the console. The cassette may include an electronic connection that interfaces with a console deliver power to the handpiece, and optionally to identify characteristics such as probe and/or handpiece type. The cassette may further include a flow correction circuit for reducing unwanted flow blockages or vacuum.

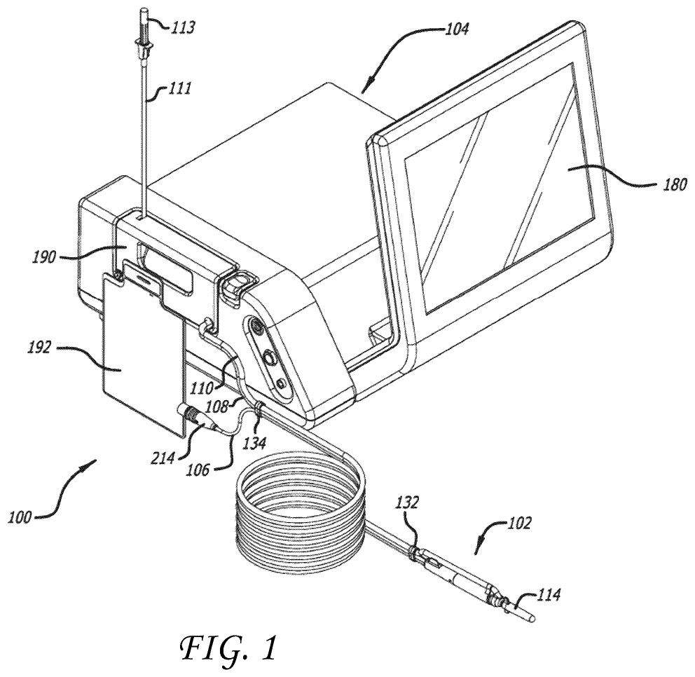

FIG. 1 illustrates an example system according to one implementation of the present disclosure that is configured to percutaneously access and act upon target tissue while helping reduce collateral trauma. In some example implementations, the minimally-invasive ultrasonic nature of system 100 increases the accuracy of removing diseased tissue when compared to surgical procedures which include surgical dissections of healthy tissue. In some implementations, the percutaneous, minimally-invasive nature of system 100 facilitates treatment of a patient in an office setting under local anesthesia. Treatment in an office setting is advantageous in several respects, for example, including patient comfort and convenience and avoiding costs associated with operating room time and general anesthesia.

In some implementations, as illustrated in FIG. 1, system 100 includes delivery device 102 and controller 104 which may be operatively connected delivery device 102. In some implementations, the delivery device 102 may be operatively connected to controller 104 via power line 106, aspiration or vacuum line 108, and irrigation line 110. Power line 106 may connect to controller 104 via a wired connection as shown in FIG. 1. In some implementations, power line 106 may connect to controller 104 via a power line connector 214. In some implementations, controller 104 may be configured to communicate with delivery device 102 via a wireless communication or a combination of a wired communication and a wireless communication.

In some implementations, delivery device 102 may be configured to transmit ultrasonic energy to a percutaneous musculoskeletal site at a pre-tuned frequency selected to debride musculoskeletal tissue. As illustrated in FIG. 1, in some implementations, delivery device 102 includes a cap 114. Generally, various components of delivery device 102 contemplated for tissue contact are formed of biocompatible and/or other suitable materials. As illustrated in FIG. 1, delivery device 102 may be ergonomically designed, adapted to be hand-held (e.g., as a stylet), or otherwise adapted to be manually operated using a single hand. In some implementations, the delivery device 102 may be adapted to be manipulated automatically or semi-automatically (e.g., as part of a robotic system).

In certain implementations, delivery device 102 may be pre-tuned to a selected ultrasonic energy frequency or frequency range. For example, an ultrasonic energy frequency range from about 25 kHz to about 29 kHz effectively debrides pathologic musculoskeletal tissue (e.g., scar tissue associated with a tendon) while reducing the likelihood of trauma to healthy soft tissue.

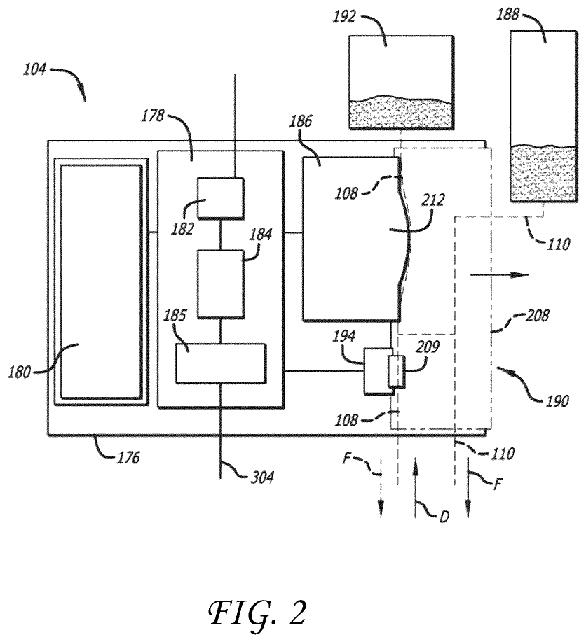

As illustrated in FIGS. 1 and 2, in some implementations, system 100 may include a reusable controller 104 connected to a disposable cassette 190 and disposable collector 192. As illustrated in FIG. 2, a reusable controller 104 may include: (a) housing 176; (b) command module 178 including: (i) power source 182; (ii) processor 184; and (iii) signal filter 185; (c) vacuum source 186; and (d) irrigation source 188. In some implementations, vacuum source 186 and irrigation source 188 each include a pump motor. In some implementations, vacuum source 186 and irrigation source 188 include a single pump motor. In some implementations, command module 178 includes a main unit which preferably includes one or more processors electrically coupled by an address/data bus to one or more memory devices, other computer circuitry, and one or more interface circuits. The processor may be any suitable processor and may include volatile memory and non-volatile memory. In some implementations, the memory stores one or more software programs that interacts with the other devices in system 100. These programs may be executed by the processor in any suitable manner. In an example implementation, the memory may be part of a “cloud” such that cloud computing may be utilized by system 100. The memory may also store digital data indicative of documents, files, programs, web pages, etc. retrieved from a computing device and/or loaded via an input device.

In particular implementations, command module 178 may be configured to control flow from vacuum source 186 and/or from irrigation source 188. In some implementations, command module 178 may be configured to power delivery device 102. In some implementations, command module 178 may be configured to provide instructions to a user via and/or enable a user to select instructions, for example, via user interface 180. In some implementations, command module 178 includes signal filter 185 for delivering a conditioned power signal (e.g., a sinusoidal power signal at a selected amplitude and frequency) to delivery device 102.

As further illustrated in FIG. 2, command module 178 may include at least one processor 184. In some implementations, controller 104 includes user interface 180. User interface 180 may include a touch screen system for controlling system 100. In some implementations, controller 104 includes power source 182. Power source 182 may include a battery, a capacitor, a transformer connected to an external power source, such as a wall socket, combinations thereof, or other means for providing electrical power to system 100. Power source 182 may also directly or indirectly deliver power to various components of controller 104 as appropriate.

In some implementations, controller 104 includes vacuum source 186. Vacuum source 186 may be a peristaltic pump. In some implementations, vacuum source 186 may be a pump motor for operating a diaphragm in a connected disposable cassette 190.

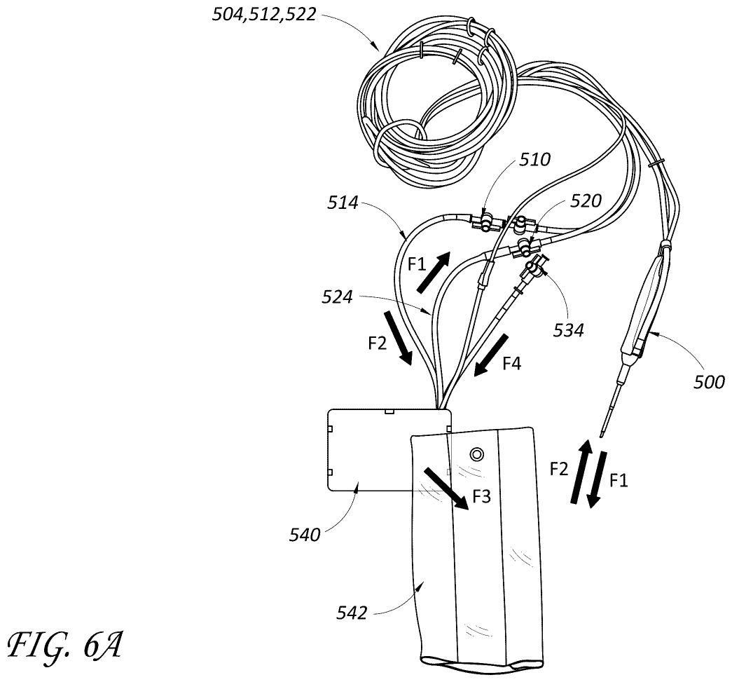

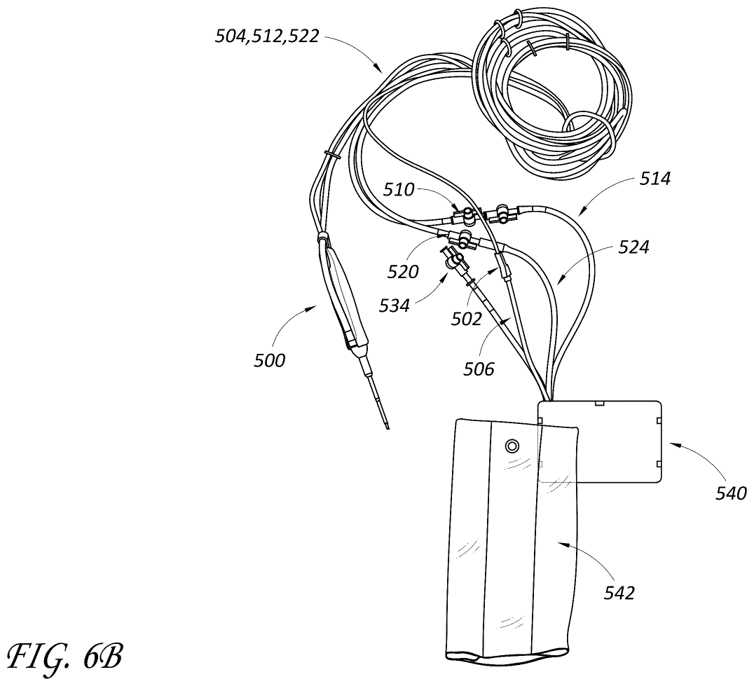

Reusable controller 104 may removably receive a connected disposable cassette 190. In some implementations, such as system 100 illustrated in FIG. 1, disposable cassette 190 may include an administration line 111 with a spike 113 that may be operatively coupled to administration line 111. In some implementations, cassette 190 includes collector 192. Collector 192 may be configured to receive detritus, fluid, or other matter being aspirated by the aspiration flow D. Collector 192 may be a bag or other container. As illustrated in FIGS. 1 and 2, collector 192 may be separate from cassette 190. In other implementations, collector 192 may be maintained by, formed as a part of, or a component within cassette 190. In some implementations, such as the cassettes shown in FIGS. 6A-6B discussed below, the collector 192 may be configured to removably connect to a cassette 190, such as with a snap connection, magnetic connection, screw, luer, and the like. In some implementations, collector 192 may be connected to the cassette 190 using double sided tape.

In some implementations, controller 104 may include irrigation source 188. Irrigation source 188 may include a reservoir of irrigant (e.g., saline). In some implementations, the reservoir may be pressurized by gravity, a plunger (e.g., a syringe), and/or a pump (e.g., a peristaltic pump operated by controller 104 and optionally disposed within the housing 176) to generate fluid flow F. In some implementations, the irrigation source 188 may be separate from the system 100. In some implementations, spike 113 may be configured to penetrate the separate irrigation source to supply fluid flow to system 100. In some implementations, controller 104 includes valve actuator 194, which may be configured to direct fluid flow F into vacuum conduits of delivery device 102, for example for flushing purposes. In some implementations, irrigation source 188 may be a peristaltic pump. In some implementations, irrigation source 188 may be a pump for operating a diaphragm in a connected disposable cassette 190. In some implementations, vacuum source 186 discussed above may be combined with irrigation source 188, for example as a single pump motor for operating two diaphragms in a connected disposable cassette 190.

In some implementations, a user interface 180 can include buttons for a prime phase, a purge phase and/or a reset phase. In some implementations, the user interface 180 enables a sequential operation of delivery device 102 starting with an ultrasound level selection, irrigation level selection, and aspiration level selection. In some implementation, the user interface may display ultrasound images as the handpiece debrides the diseased site. The user may be allowed to select the various levels in sequence when operating system 100. In some implementations, levels and/or sequence parameters may be illuminated and/or required sequentially. For example, in some implementations, a user may not be enabled to make a subsequent selection until the previous selection(s) are finalized.

In some implementations, ultrasound energy, irrigant flow, and aspiration flow are delivered and/or controlled independently. In some implementations of operation, these features can be coupled together for delivery. For example, ultrasound energy and irrigant can be delivered concurrently, while aspiration flow may be delivered intermittently. In some implementations, the ultrasound energy and irrigant flow may optionally cease during aspiration and can be restarted once treatment may be reinitiated. In some implementations, irrigant flow may cease while ultrasound energy continues during aspiration, although some of the beneficial effects from using irrigant during ultrasonic treatment (e.g., continuous tip cooling and tissue emulsification, as well as others) are potentially reduced by such operation. In some implementations, the ultrasound energy, irrigant flow, and aspiration flow can be periodically or temporarily coupled and/or uncoupled throughout a procedure.

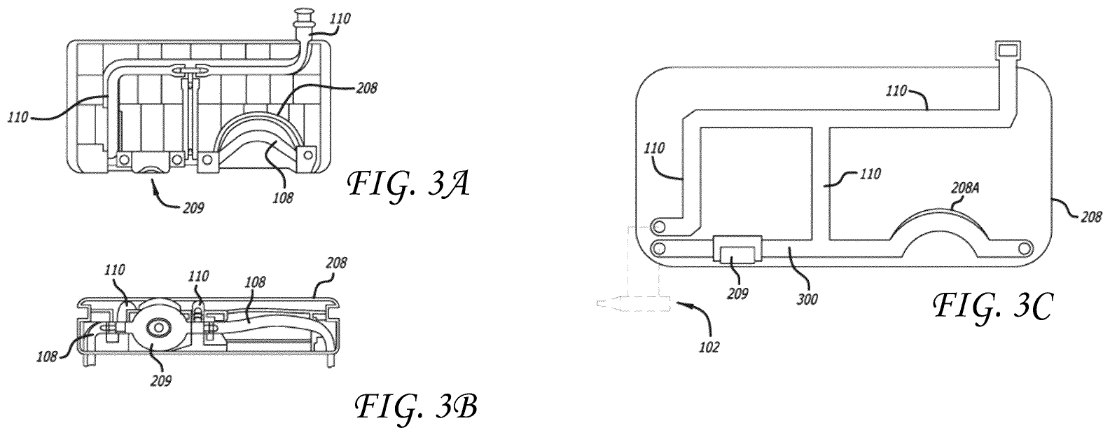

In some implementations, as illustrated in FIGS. 2 and 3A-3C, a cassette 190 includes: (a) housing 208; (b) valve 209; (c) a portion of the vacuum line 108; and (d) a portion of irrigation line 110 (designated by broken lines). In some implementations, vacuum line 108 and irrigation line 110 include a plurality of interconnected segments of medical tubing, although unitary constructs are a potential option as well. A cassette 190 may connect vacuum line 108 to vacuum source 186 in a relatively sterile manner. For example, where vacuum source 186 includes a peristaltic pump, tubing cassette 190 includes seat structure 210 for causing vacuum line 108 to engage pump drive 212 of vacuum source 186 that generates aspiration flow in vacuum line 108.

FIGS. 3A-3C illustrate an example cassette 190. FIG. 3A shows an interior side of cassette 190, FIG. 3B illustrates an example bottom side of cassette 190, and FIG. 3C is a schematic view of an example cassette 190. In some implementations, vacuum line 108 and irrigation line 110 may be referred to as a tubing set. In operation of one example implementation, pump drive 212 of vacuum source 186 (e.g., a peristaltic pump) may be received in seat structure 210 such that vacuum line 108 is engaged against seat structure 210 between the pump drive 212 and seat structure 210. Valve 209 may be engaged by valve actuator 194 to press valve 209 closed such that flow from irrigation line 110 will not travel through vacuum line 108 to delivery device 102 (see FIG. 2). In some implementations, valve 209 can be released to enable fluid to flow into vacuum line 108 to the device and through the vacuum conduits, for example to flush a vacuum line 108. In some implementations, the irrigant flowing through irrigation line 110 is optionally gravity pressurized or otherwise forced through system 100.

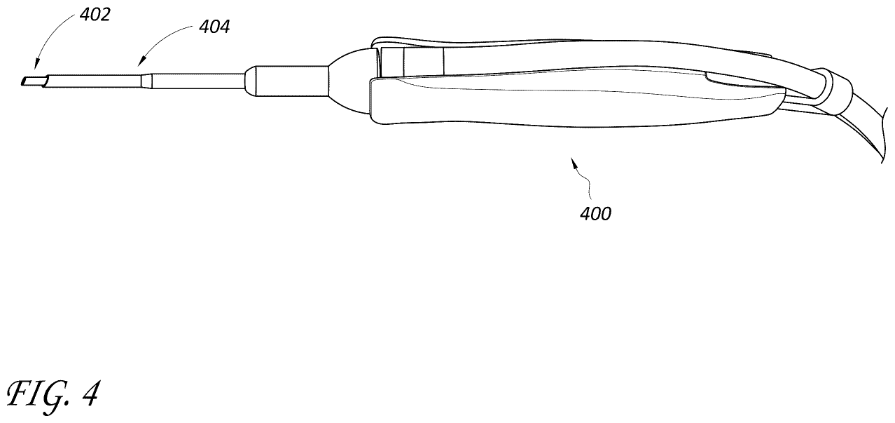

In some implementations, the delivery device 102 is a disposable delivery device, for example disposable delivery device 400 shown in FIG. 4. The delivery device 400 can include an aspiration or vacuum tube 402 and an irrigation tube 404. In some implementations, such as delivery device 400, the vacuum tube 402 may be an inner tube and the irrigation tube 404 may be an outer tube. In some implementations, the vacuum tube 402 may be an outer tube and the irrigation tube 404 may be an inner tube. In certain implementations, the vacuum tube 402 and irrigation tube 404 are arranged longitudinally, for example, side-by-side or one on top of the other. In some implementations, the vacuum tube 402 and irrigation tube 404 are a single tube, for example one tube controlled with a valve for aspiration and irrigation functions, or one tube with multiple lumens for each function. One of skill in the art will understand that other potential arrangements may be suitable.

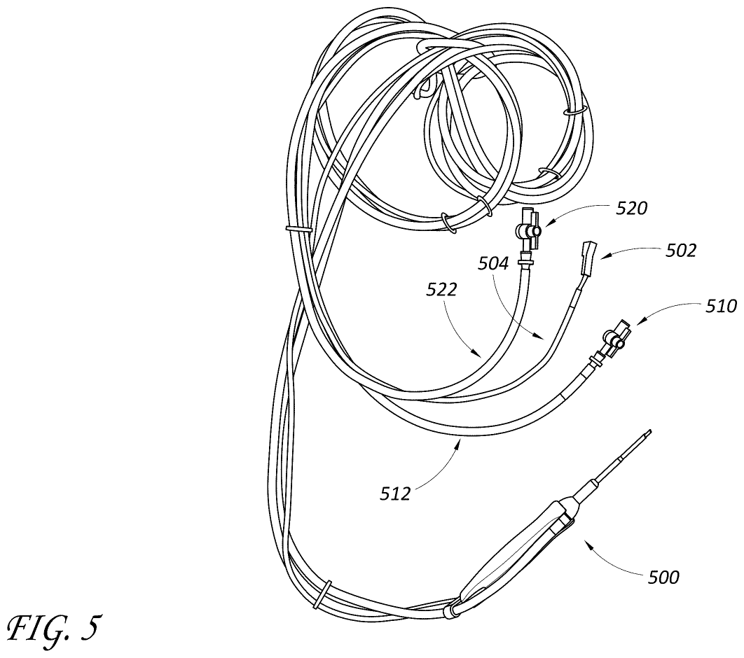

As shown in FIG. 5, a disposable delivery device 500 can include multiple proximal connectors. For example, in some implementations, disposable delivery device 500 can include a power connection 502, an aspiration connection 510, and an irrigation connection 520. In some implementations, aspiration connection 510 can be connected to delivery device 500 via an aspiration tubing 512. In some implementations, irrigation connection 520 can be connected to delivery device 500 via an irrigation tubing 522. In some implementations, power connection 502 can be connected to delivery device 500 via cable 504 for delivery of ultrasonic or other signals. Some or all of the tubing 512 and 522 and/or cable 504 may be combined in a single tubing and/or connection. In some implementations, some or all of tubing 512, tubing 522, and cable 504 can be bundled together. In certain examples, some or all of the connections 502, 510, 520 can be combined in a single connection and/or connector harness.

As shown in FIGS. 6A-6B, a disposable delivery device 500 can be releasably connected to a cassette 540. For example, as illustrated, disposable delivery device 500 power connection 502 can connect to a power supply line 506. Aspiration connection 510 can connect to aspiration line 514, for example to provide aspiration flow F2 from the handpiece 500. Irrigation connection 520 can connect to irrigation line 524, for example to provide irrigation flow F1. An additional tubing 534 with an optional connector can be used to connect the cassette 540 with an irrigation source, for example provide a flow F4 of irrigant such as saline. In some implementations, the fluid and power connections are made with any suitable connector. For example, the power connections can be made with one or more cables and/or switches, and the fluid connections can be made with one or more tubes and/or valves, such as luer, backflow, and/or controllable valves. In some implementations, some or all of the tubing 514, 524, and 534 and/or power supply line 506 can be combined in a single tubing and/or connection. In some implementations, some or all of tubing 514, tubing 524, tubing 534 and power supply line 506 can be bundled together.

In some implementations, cassette 540 includes the ability store material, for example, material collected from the aspirating handpiece 500 for disposal and/or analysis. In some implementations, material is collected in a collector 542, for example, a collection bag or other flexible storage means as shown in FIGS. 6A-6B. In some implementations, a collector 542 may be attached or connected to the cassette 540 to receive a flow F3 of aspirated material from the cassette 540. In some implementations, the collector 542 may be a canister, reservoir, or other rigid storage means. In some implementations, the collector 542 may be releasably connected to the cassette 540, as discussed in further detail below. In some implementations, the reservoir 542 may be integrated with the inside or outside of the cassette 540 (for example, the collector 192 discussed above with respect to FIG. 1).

In certain implementations, a plurality of disposable delivery devices, such as the delivery device 500 are provided with corresponding disposable cassettes, such as cassettes 190, 540 for each delivery device. Individually pre-tuning the devices to an appropriate ultrasonic energy frequency, such as that previously described, before delivery to the user removes a need to test and adjust power signal parameters or delivery device configurations prior to or during each procedure. In some implementations, a single use cassette/delivery device kit is set up or configured prior to delivery to the end user. This kit may be used in a treatment procedure, and is optionally discarded at the end of the procedure, thereby reducing operation time, a requisite skill level for “tuning” system 100, and/or additional components or systems for tuning delivery device 500. Moreover, the combination of cassette 190 and delivery device 102, delivery device 500 and cassette 540, etc. eliminates a need to sterilize equipment before a procedure, as all components that come into contact with bodily fluids are pre-sterilized and discarded at the end of the procedure.

In some implementations, after tissue treatment is completed, cassette 540 may be removed from controller 104, discarded, and replaced with a second, sterile cassette, which may be either pre-connected or subsequently connected to a second, sterile delivery device (not shown) to sterilize the system 100 for a new procedure.

In some implementations, the disposable portion including handpiece 500, cassette 540, and collector 542, may be connected as discussed above and as shown in FIGS. 6A-6B. As illustrated in FIG. 6A, the disposable portion may provide one or more fluid flow paths. In some implementations, irrigant such as saline or an IV bag may be connected to irrigation tubing 534 to provide a flow F4 of irrigant. The irrigant flow F4 enters the cassette 540 where it may be pumped as described below. Irrigant flow F1 exits the cassette 540 through irrigation line 524 and connector 520, to irrigation tubing 522 of the handpiece 500, where it can be used to irrigate the handpiece 500 and/or treated tissue. Similarly, aspiration flow F2 may be provided from the handpiece 500, through aspiration tubing 512 of the handpiece 500, to connector 510 and aspiration line 514 into cassette 540 where it may be pumped as described below. Cassette 540 may pump the aspirated material to provide aspirated material flow F3 from the cassette 540 to the collector 542.

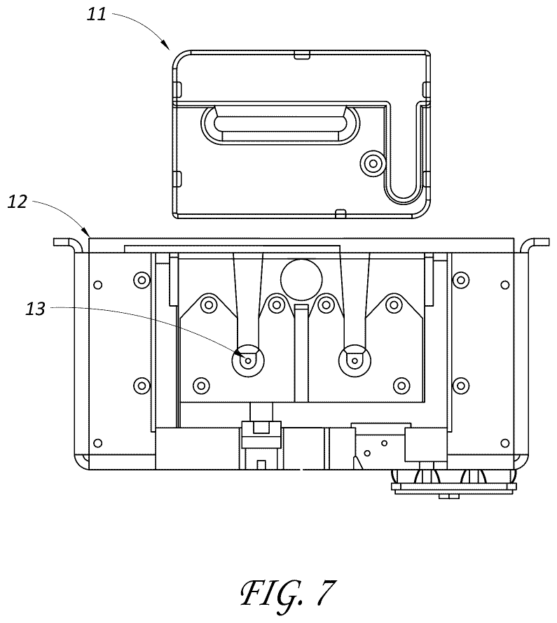

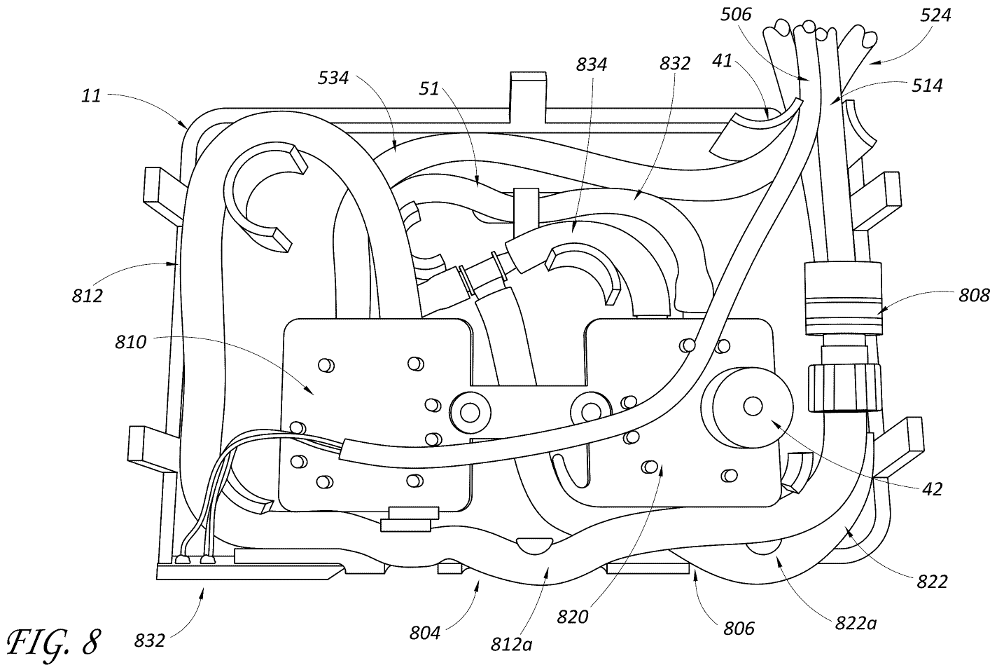

FIGS. 7-16 illustrate various features of alternate implementations of the cassette 540. As shown in FIG. 7, disposable cassette 11 may interface with a console 12. In certain examples, the interface may include includes a pump connection 13 to at least one fluid pump in the console 12. The pump connection 13 may include a mechanism that transmits motion from a pump component in the console 12 to a mating pump component in the cassette 11. For example, pump connection 13 can connect a pump diaphragm in disposable cassette 11 to a diaphragm pump motor housed in the console 12. FIG. 8 shows an internal arrangement of the disposable cassette 11. In some implementations, two pumping interfaces, such as irrigation pump area 810 and aspiration pump area 820 shown in FIG. 8, each use pump connection 13 to mate with respective pump motors housed in the console 12. In some implementations, pump connection 13 is a single connection. As shown, a first pump, for example irrigation pump area 810, uses pump connection 13 to mate with a diaphragm pump motor in the console 12. This arrangement allows body-contacting components and fluids to be contained within the disposable section including the cassette 11, collector, handpiece 500, and tubing as discussed below, while keeping the reusable section including console 12 free of any body-contacting materials and/or fluids.

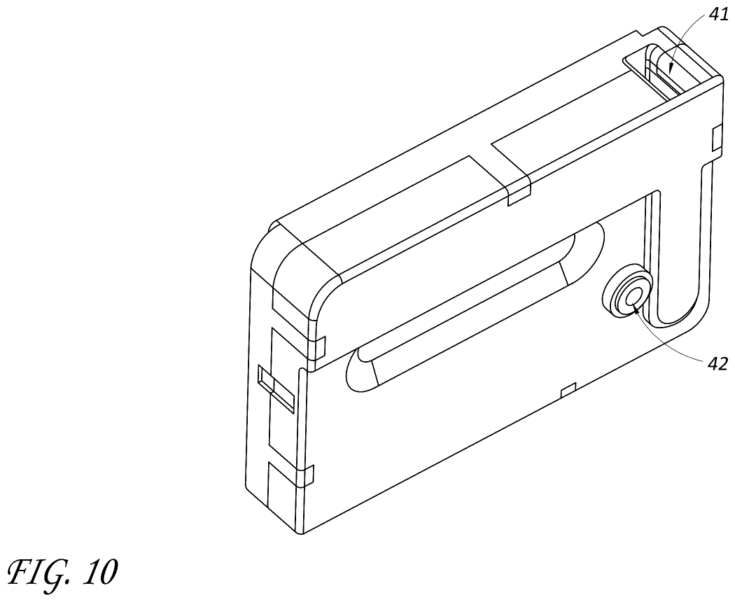

Referring to FIGS. 8, 15, 16A, and 16B, in some implementations, the cassette 11 can include an irrigation circuit to move fluid, for example irrigation fluid such as saline, from an external connection (not shown) to the handpiece. In some implementations, irrigation fluid is pumped through irrigation tubing 534, through irrigation pump area 810, into an internal irrigation line 812. In some implementations, irrigation line 812 exits the housing of the cassette 11 at an opening 804 to create an exposed section 812a, as discussed below. Irrigation line 812 then enters connector 808 to connect to irrigation tubing 524, which exits the cassette 11 at opening 41 (see FIG. 10) for connection to the handpiece as shown in FIGS. 6A-6B.

Still referring to FIGS. 8, 15, 16A, and 16B, in some implementations, cassette 11 can include an aspiration circuit to move fluid from the handpiece to a collector. For example, in some implementations, aspiration tubing 514 from a handpiece (see FIGS. 6A-6B) enters the cassette 11 at opening 41 (see FIG. 10). Aspiration tubing 514 connects to the internal aspiration line 822 via connector 808. In some implementations, aspiration line 822 exits the housing of the cassette 11 at an opening 806 to create an exposed section 822a, as discussed below. Aspiration line 822 then connects through the aspiration pump area 820 to the outlet 42 (see FIG. 10). In some implementations, outlet 42 may be connected to an internal collector as discussed above. In some implementations, outlet 42 may be releasably connected to a collector, such as bag collector 542 shown in FIGS. 6A-6B. The aspiration circuit may be used to allow the cassette 11 to move fluid, for example, excess irrigation fluid, waste, and/or detritus from a handpiece to a collector.

In some implementations, the aspiration circuit and/or irrigation circuit may include one or more filters. The filters can be located within the flow circuits to prevent detritus, particles, or other contaminates from reaching protected locations. For example, in some implementations, a filter or series of filters may be used to prevent contaminates from reaching a sensor, as discussed below. In some implementations, a filter or series of filters may be used to prevent aspirated detritus from reaching the aspiration pump area. Similarly, a filter or series of filters may be used to prevent external contaminates in the irrigant line, for example particles accidentally introduced during connection of a saline source, from reaching the irrigation pump area and/or the handpiece 500. In some implementations, the filter may be accessible for cleaning and/or replacement.

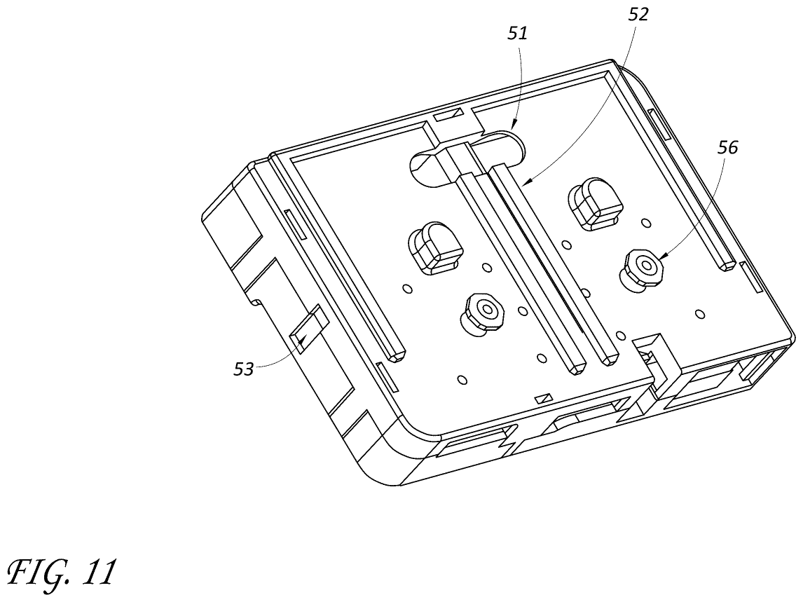

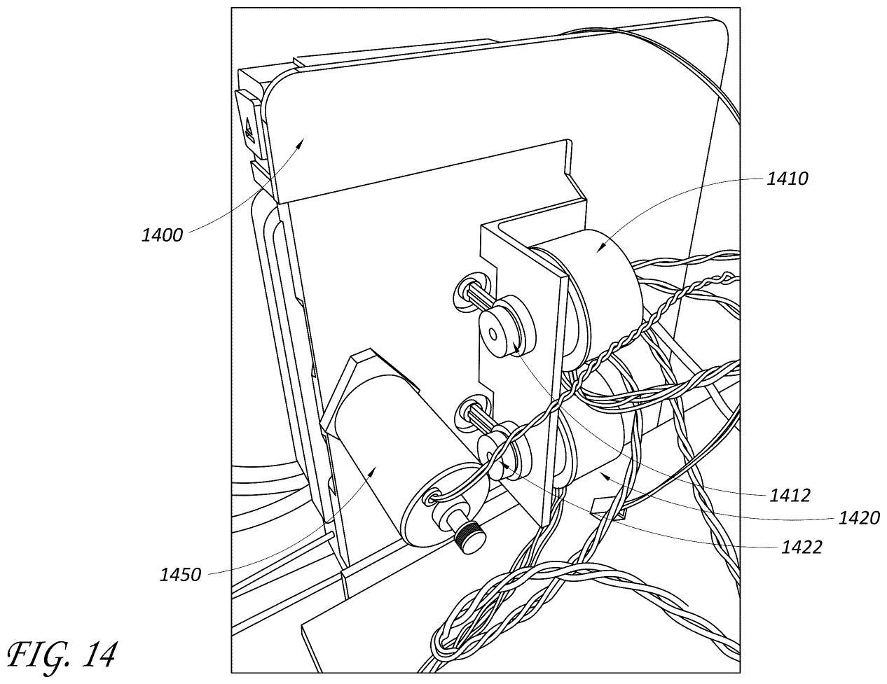

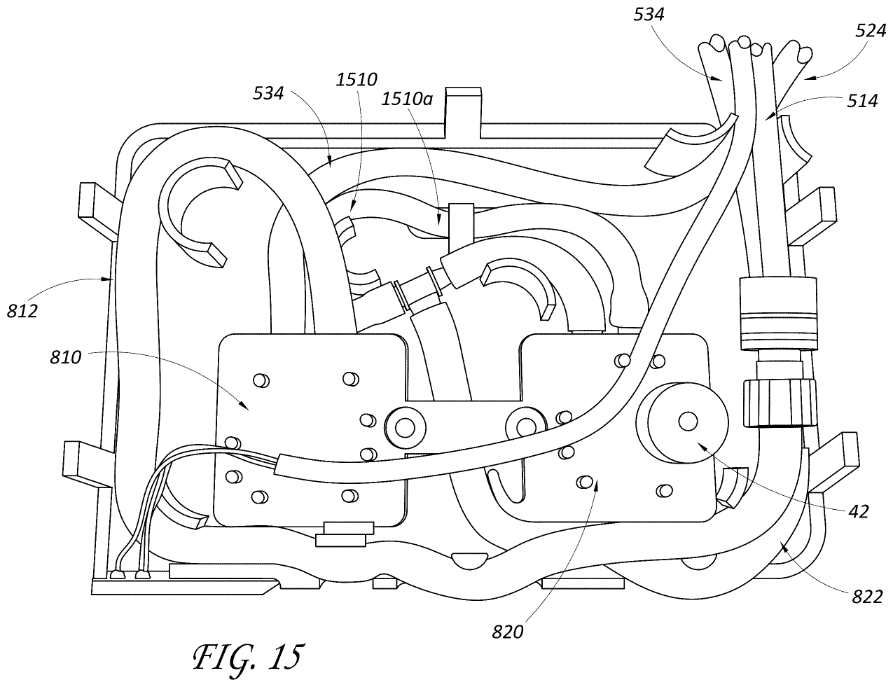

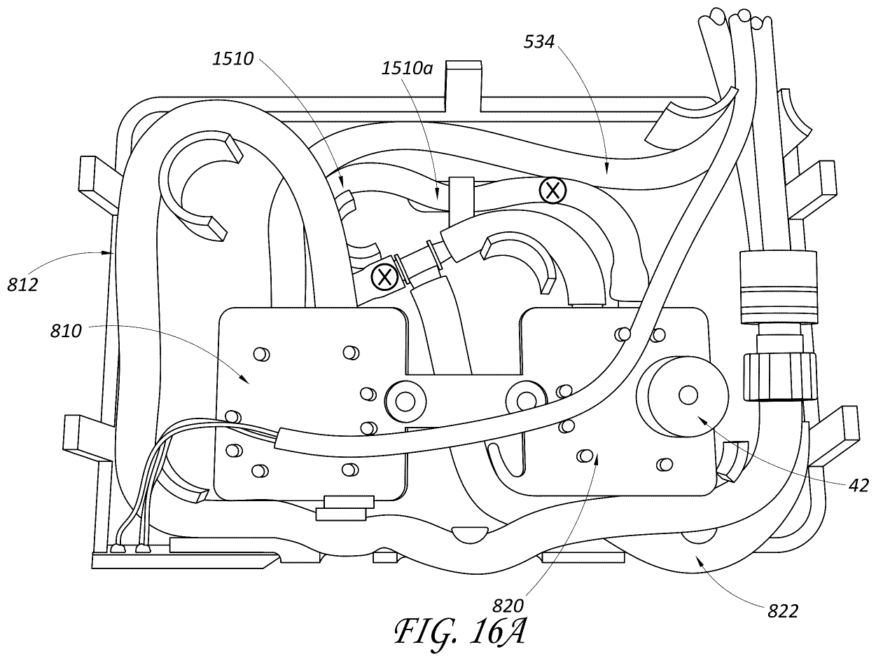

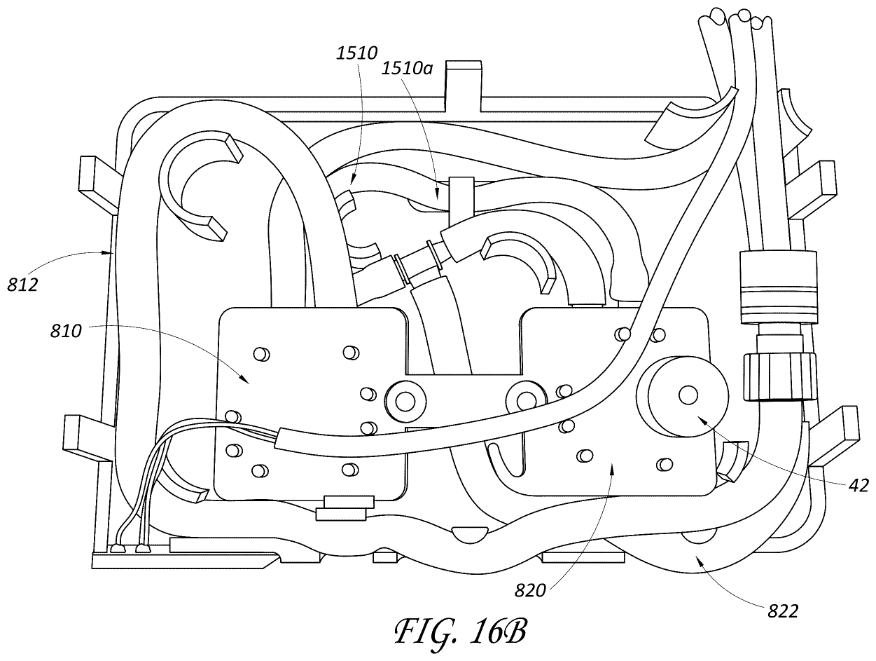

Still referring to FIGS. 8, 15, 16A, and 16B, in some implementations, cassette 11 can include a flow corrector. As shown in FIG. 15, a flow correction circuit can include a secondary flow pathway 1510. Secondary flow pathway 1510 can include an exposed section 1510a that may be accessible through opening 51 (see FIG. 11). Exposed section 1510a mates with a solenoid pinch valve 1450, shown in FIG. 14. The pinch valve 1450 may be normally closed as illustrated in FIG. 16A, and keeps the secondary flow pathway 1510 closed. This arrangement only allows flow of aspirated material into the cassette 11 and out to the collector 542 through outlet 42. Occasionally, the handpiece 400 may become occluded, usually by a build-up of tissue or a large chunk of material blocking the needle opening. If the handpiece 400 becomes occluded, the vacuum will build up and the occlusion cannot release. At this point, the sensors, such as the aspiration pressure sensor discussed below, can detect the pressure increase and the pinch valve 1450 releases to open the secondary pathway 1510 as shown in FIG. 16B. The secondary pathway 1510 recirculates to and from the outlet 42 and collector 542, temporarily preventing flow in the aspiration line 822. The outlet 42 then also becomes an inlet. By maintaining the inlet/outlet 42 into the collector 542 above where the fluid collects, fluid can be prevented from recirculating from the collector 542 back into the aspiration pumping circuit. The collector 542 can be provided with an opening for air, which allows air inflow to the cassette 11 and a break of the vacuum pressure. Once vacuum is broken, the occlusion will release from the tip of the handpiece, the pinch valve 1450 will close again, and flow will resume through the normal aspiration pathway as shown in FIGS. 15 and 16A.

As further illustrated in FIG. 8, in some implementations, a cassette 11 can include a circuit board 832 connected to a power supply line 506. Power supply line 506 can be connected to power connection 502 to operate a handpiece 500. Power supply line 506 can be used to deliver control signals and/or power to operate the ultrasonic cutting of the handpiece 500. In some implementations, the power supply line 506 can also enable two-way communication to the handpiece 500 for feedback. In some implementations, power supply line 506 may be connected to a circuit board 832 in the cassette 11. In some implementations, circuit board 832 connects to the console 12 when the cassette 11 is connected. In some implementations, circuit board 832 may be separately connected to the console 12 via wired and/or wireless connection.

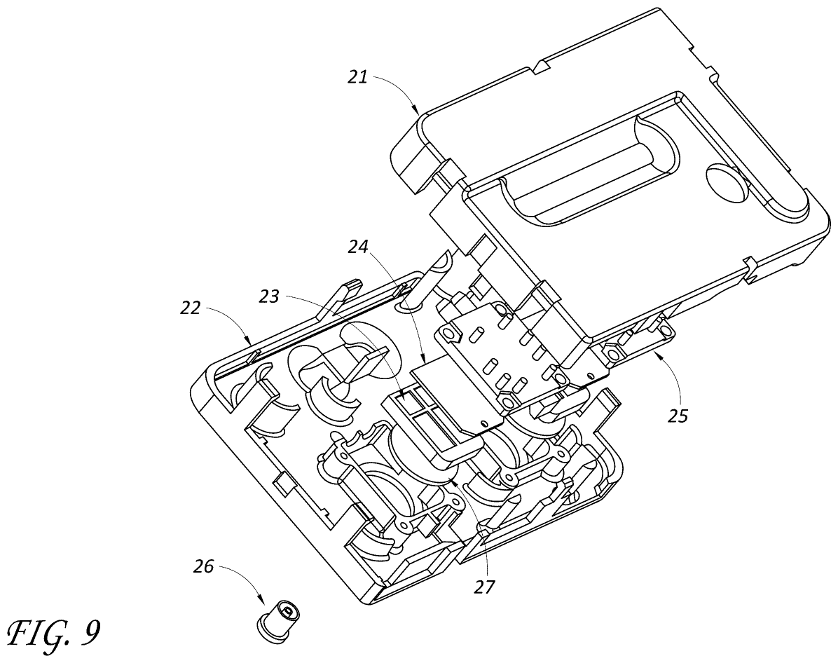

FIG. 9 shows an exploded view of an implementation of the cassette 11, where the various fluid and electrical lines are omitted for clarity. As shown, the cassette can include a front housing 21 that mates with a back housing 22 to enclose some or all of the various components discussed above. As shown, the cassette 11 can combine both an irrigation pump area 810 and an aspiration pump area 820 into a valve and diaphragm block 23. The valve and diaphragm block 23 can compress the valves and diaphragm to create a fluid-tight seal when the front housing 21 and back housing 22 are engaged and the cassette 11 is inserted in the console 12. A valve gasket 24 can surround the valve and diaphragm block 23 to improve sealing. A valve block 25 can compress valves at the outlet 42 to create a seal and fluidically connect a handpiece 400 to a collector, for example collector 542, via the surgical tubing circuits. Diaphragm 27 within a pump area, for example irrigation pump area 810 and/or aspiration pump area 820, mates with one or more diaphragm pumps or pump motors in the console 12 via diaphragm connector 26. In some implementations, respective diaphragms 27 and diaphragm connectors 26 are used for each of the irrigation pump area 810 and aspiration pump area 820 connections. In some implementations, the pump areas 810, 820, including pump motors, diaphragms 27, connectors 26, or other components can be balanced to provide approximately equal aspiration and irrigation flow. In some implementations, the pump areas 810, 820 may be dynamically adjusted, for example by dynamically controlling the operating rate of the pump motors within the console 12.

FIG. 11 illustrates the back, or console-facing side, of an implementation of a cassette 11. The diaphragm connectors 56 may be similar to diaphragm connectors 26 discussed above in some or all respects. As shown, in some implementations, diaphragm connectors 56 are exposed on the back of the cassette 11 to interface with motors in a console 12. The console motors can be activated to actuate diaphragms, for example diaphragms 27, in the cassette 11 to create a diaphragm pump for irrigation pump area 810 and aspiration pump area 820 as discussed above. FIG. 11 also shows rails 52, which can be used in some implementations to engage the cassette 11 with the console 12. For example, rails 52 can assist with insertion of the cassette 11 into the mating connection area of the console 12, and can help prevent crooked and/or upside-down placement. Cassette 11 may include rails 52 in the center, as illustrated in FIG. 11. Some implementations of cassette 11 may additionally or alternatively include rails 52 on the perimeter of the back of the cassette 11, on the front of the cassette 11, and/or any of the sides of the cassette 11. In some implementations, the side of the cassette 11 may include an engagement member, for example a detent 53 for a locking latch. In some implementations, the engagement member may include a release such as a button, lever, slide, and the like to releasably secure the cassette 11 in the console 12. In some implementations, the engagement member may be a frangible clip that must be broken or otherwise disabled to remove a cassette 11 from a console 12. This feature can prevent reuse of a removed cassette 11, thereby effectively making a cassette 11 into a single-use disposable cassette 11.

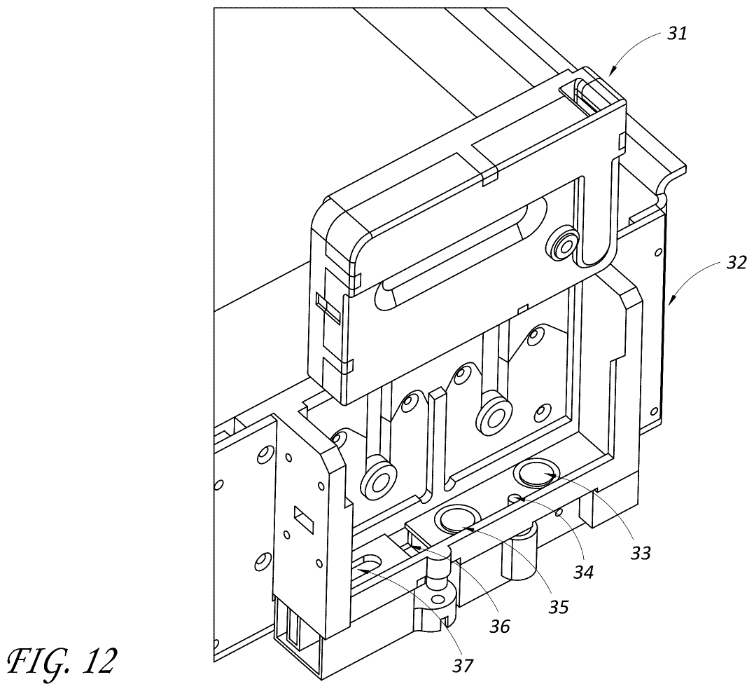

FIG. 12 illustrates a cassette 31, which may be similar to cassette 11 in some or all respects. As shown, cassette 31 interfaces with a console 32, which includes various sensors. For example, in some implementations, a console 32 can include a pressure sensor for aspiration 33, a cassette placement sensor 34, a pressure sensor for irrigation 35, a bubble detection sensor 36 and/or a cassette identification sensor 37. In some implementations, the sensors can be used to generate alerts and/or warnings of improper or undesirable conditions. In some implementations, the sensors can be used to automatically stop or prevent system operation, or to automatically enable and/or control system operation. In some implementations, the sensor can use a sensor threshold to establish safe operating parameters. In some implementations, some or all of the sensors may be combined, for example a combined pressure sensor for detecting pressure in both the aspiration and irrigation circuit, a combined pressure and bubble sensor, or other appropriate combination. In some implementations, the irrigation and/or aspiration circuits may include a flow sensor. In some implementations, some or all of the pressure sensor for aspiration 33, cassette placement sensor 34, pressure sensor for irrigation 35, bubble detection sensor 36, cassette identification sensor 37, or other sensor may be included in the cassette 31 and interface with the console 32 via appropriate connector.

The pressure sensor for aspiration 33 and the pressure sensor for irrigation 35 may be similar in some or all respects. The pressure sensors 33, 35 can be used to measure fluid pressure within the respective aspiration line and irrigation line. In some implementations, such as the cassette 11 illustrated in FIG. 8, the aspiration line 822 and irrigation line 812 within the cassette 11 include respective exposed sections 822a and 812a. These sections 822a, 812a mate with a pressure sensor 33, 35, when the cassette 11 (or 31) is seated in the console 12 (or 32). In some implementations, the aspiration line 822 includes a tube for aspirated material as discussed above. The exposed section 822a may be compressed onto a pressure sensor for aspiration 33. As vacuum from the aspiration pump increases, the aspiration tube section 822a will contract in diameter and decrease the pressure on the sensor 33. The pressure changes can be used to detect occlusions of the handpiece tip, which will result in increasing vacuum until the occlusion is removed. Similarly, in some implementations, the irrigation line 812 includes a tube for irrigated fluid as discussed above. An exposed section 812a may be compressed onto a pressure sensor for irrigation 35. As fluid pressure increases, the irrigation tube section 812a will expand in diameter and increase the pressure on the sensor 35. The pressure changes can be used to detect if there is flow through the irrigation tube 812, which signifies whether or not the irrigation fluid source, for example an IV bag, is empty or filled. In some implementations, some or all of the tubing in the cassette that interfaces with a pressure sensor may be protected with a filter. For example, a filter may be included in the fluid flow path at or before exposed sections 822a and/or 812. In some implementations, a filter may be included in the fluid flow path before the pressure sensor, for example, when the sensors are located within the cassette 11.

In some implementations, the cassette placement sensor 34 can detect proper placement of a cassette 11, 31 in a console 12, 32. In some implementations, a cassette placement sensor 34 may be a switch that is activated by a spring-loaded pin. When the cassette 31 is properly placed into the console 32, it depresses a spring-loaded pin, which pushes a lever on the switch. When the switch is activated, it signifies that the cassette 31 is properly located. In some implementations, the cassette placement sensor 34 includes multiple spring-loaded pins, each with a respective switch. This arrangement can advantageously detect proper alignment of an inserted cassette 31. In some implementations, the cassette placement sensor 34 can include electrical switches that must be engaged before the system can be used, for example automatically locking the system until a cassette 31 is properly inserted. In some implementations, the cassette placement sensor 34 can trigger an alert or other warning if a cassette 31 is not properly inserted.

In some implementations, the bubble detection sensor 36 can be used to detect the presence of an air bubble. In some implementations, the irrigation fluid source may contain bubbles, for example there may air pockets in an IV bag used as a source of saline irrigation fluid. The irrigation pressure sensor 35 discussed above can be used to measure irrigation fluid flow and can detect pressure change but cannot identify a cause of the detected pressure. Therefore, there may be instances when the irrigation pathway 812 pumps air or other gas, rather than the intended irrigation liquid. The bubble detection sensor 36 may be able differentiate gas (air) and liquid (saline) through the irrigation tube 812, which can alert the user they are not irrigating correctly. In some implementations, some or all of the tubing in the cassette that interfaces with a bubble sensor may be protected with a filter. For example, a filter may be included in the fluid flow path at or before exposed sections 822a and/or 812. In some implementations, a filter may be included in the fluid flow path before the bubble sensor, for example, when the sensor is located within the cassette 11.

In some implementations, the cassette identification sensor 37 can be used to identify a variety of system attributes. In some implementations, a PCB, for example circuit board 832 inside the cassette 11, interacts with pogo pins inside the console 32. When the pins make contact with pads on the PCB, the console 32 will be able to detect information for the cassette 31. For example, the cassette identification sensor 37 can be used to identify a cassette type, cassette manufacturer, irrigation fluid type, sterilization status, number of uses, expiration dates, handpiece type and/or size, and/or clinician operator. This information can be encoded in the pins discussed above, or via other hardware encoding. In some implementations, the information may be encoded via software that is read by the cassette identification sensor 37 and/or the console 32. In some implementations, the cassette identification sensor 37 can be included on a circuit board that is also used to interface with a cassette circuit board, such as circuit board 832 discussed above, to deliver power to the handpiece when the cassette 31 and handpiece 400 are connected. In some implementations, identification sensors may be included in the handpiece and/or handpiece connection. For example, a handpiece may include a handpiece type, handpiece manufacturer, irrigation fluid type, sterilization status, number of uses, expiration dates, handpiece type and/or size, and/or clinician operator and other characteristics as described above.

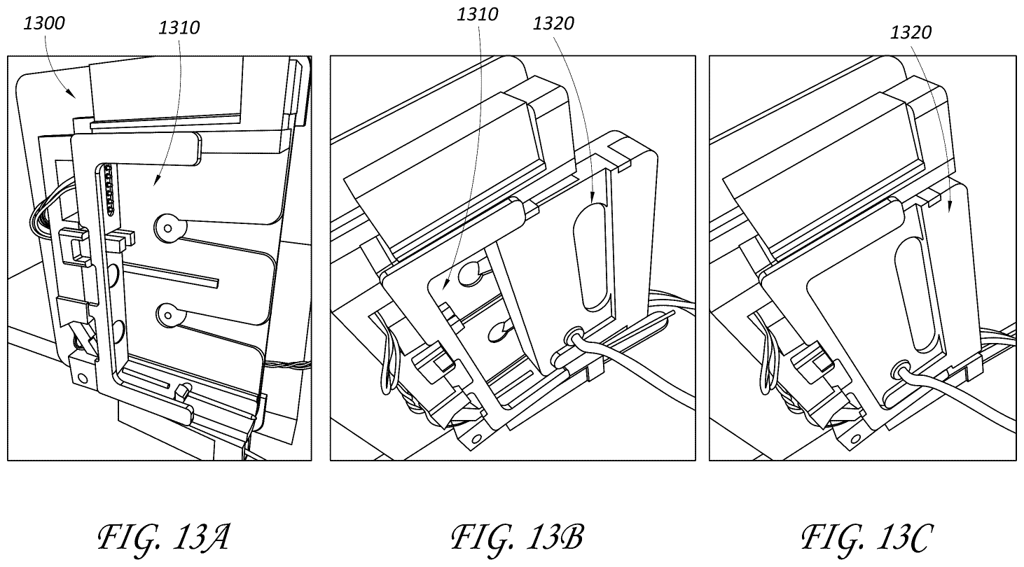

FIGS. 13A-C illustrate an implementation of a console 1300 with a cassette interface 1310. As shown in FIG. 13B, a cassette 1320 slides into the interface 1310 until it is fully seated as shown in FIG. 13C. In some implementations, the cassette 1320 slides in horizontally as shown in FIGS. 13A-C. In some implementations, the cassette 1320 slides in vertically as shown in FIGS. 7 and 12 discussed above. In some implementations, the cassette 1320 engages the interface 1310 by twisting or rotating, snapping or pushing into the console 1300, and/or combination movements such as push and twist, or slide then rotate to lock in place.

FIG. 14 illustrates an implementation of a console 1400, including an aspiration motor 1410 with a diaphragm pump connector 1412, an irrigation motor 1420 with a diaphragm pump connector 1422, and a solenoid pinch valve 1450. As discussed above, in some implementations, the diaphragm pump connectors 1412 and 1422 respectively interface with diaphragm connectors 56 on the back of a cassette 11 to allow the motors 1410 and 1420 to acuate the diaphragms 27 in the cassette 11 for fluid flow. The solenoid pinch valve 1450 operates the flow correction circuit as discussed above.

In some implementations, system 100 may be used in any of a variety of procedures. In some implementations, system 100 may be used to perform an ultrasound-guided percutaneous tenotomy. In some implementations, the handpiece, for example handpiece 500, delivers ultrasonic energy at a frequency that may be pre-selected to debride musculoskeletal tissue upon percutaneous insertion of a distal end of the handpiece into or near a target musculoskeletal tissue site. In some implementations, system 100 enables a user to identify target tissue site 300 entirely at the time of a procedure without cutting the skin of the patient. In some implementations the delivery device 102, 500 may be pre-tuned to deliver ultrasonic energy at a frequency that reduces the likelihood of trauma to healthy soft tissue while promoting debridement of the pathologic tissue. The percutaneous, minimally invasive nature of such a procedure facilitates access and treatment of such body tissue as part of an office-based procedure under local anesthesia.

Any value of a threshold, limit, duration, etc. provided herein is not intended to be absolute and, thereby, can be approximate. In addition, any threshold, limit, duration, etc. provided herein can be fixed or varied either automatically or by a user. Furthermore, as is used herein relative terminology such as exceeds, greater than, less than, etc. in relation to a reference value is intended to also encompass being equal to the reference value. For example, exceeding a reference value that is positive can encompass being equal to or greater than the reference value. In addition, as is used herein relative terminology such as exceeds, greater than, less than, etc. in relation to a reference value is intended to also encompass an inverse of the disclosed relationship, such as below, less than, greater than, etc. in relations to the reference value. Moreover, although blocks of the various processes may be described in terms of determining whether a value meets or does not meet a particular threshold, the blocks can be similarly understood, for example, in terms of a value (i) being below or above a threshold or (ii) satisfying or not satisfying a threshold.

Features, materials, characteristics, or groups described in conjunction with a particular aspect, embodiment, or example are to be understood to be applicable to any other aspect, embodiment or example described herein unless incompatible therewith. All of the features disclosed in this specification (including any accompanying claims, abstract and drawings), or all of the steps of any method or process so disclosed, may be combined in any combination, except combinations where at least some of such features or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

While certain embodiments have been described, these embodiments have been presented by way of example only, and are not intended to limit the scope of protection. Indeed, the novel methods and systems described herein may be embodied in a variety of other forms. Furthermore, various omissions, substitutions and changes in the form of the methods and systems described herein may be made. Those skilled in the art will appreciate that in some embodiments, the actual steps taken in the processes illustrated or disclosed may differ from those shown in the figures. Depending on the embodiment, certain of the steps described above may be removed, others may be added. For example, the actual steps or order of steps taken in the disclosed processes may differ from those shown in the figure. Depending on the embodiment, certain of the steps described above may be removed, others may be added. For instance, the various components illustrated in the figures may be implemented as software or firmware on a processor, controller, ASIC, FPGA, or dedicated hardware. Hardware components, such as processors, ASICs, FPGAs, and the like, can include logic circuitry. Furthermore, the features and attributes of the specific embodiments disclosed above may be combined in different ways to form additional embodiments, all of which fall within the scope of the present disclosure.

User interface screens illustrated and described herein can include additional or alternative components. These components can include menus, lists, buttons, text boxes, labels, radio buttons, scroll bars, sliders, checkboxes, combo boxes, status bars, dialog boxes, windows, and the like. User interface screens can include additional or alternative information. Components can be arranged, grouped, displayed in any suitable order.

Although the present disclosure includes certain embodiments, examples and applications, it will be understood by those skilled in the art that the present disclosure extends beyond the specifically disclosed embodiments to other alternative embodiments or uses and obvious modifications and equivalents thereof, including embodiments which do not provide all of the features and advantages set forth herein. Accordingly, the scope of the present disclosure is not intended to be limited by the specific disclosures of preferred embodiments herein, and may be defined by claims as presented herein or as presented in the future.

Conditional language, such as “can,” “could,” “might,” or “may,” unless specifically stated otherwise, or otherwise understood within the context as used, is generally intended to convey that certain embodiments include, while other embodiments do not include, certain features, elements, or steps. Thus, such conditional language is not generally intended to imply that features, elements, or steps are in any way required for one or more embodiments or that one or more embodiments necessarily include logic for deciding, with or without user input or prompting, whether these features, elements, or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree.

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

It will also be appreciated that conditional language used herein, such as, among others, “can,” “could,” “might,” “may,” “e.g.,” and the like, unless specifically stated otherwise, or otherwise understood within the context as used, is generally intended to convey that certain implementations include, while other implementations do not include, certain features, elements and/or steps. Thus, such conditional language is not generally intended to imply that features, elements and/or steps are in any way required for one or more implementations or that one or more implementations necessarily include logic for deciding, with or without author input or prompting, whether these features, elements and/or steps are included or are to be performed in any particular implementation. The terms “comprising,” “including,” “having,” and the like are synonymous and are used inclusively, in an open-ended fashion, and do not exclude additional elements, features, acts, operations, and so forth. In addition, the term “or” is used in its inclusive sense (and not in its exclusive sense) so that when used, for example, to connect a list of elements, the term “or” means one, some, or all of the elements in the list. In addition, the articles “a,” “an,” and “the” as used in this application and the appended claims are to be construed to mean “one or more” or “at least one” unless specified otherwise. Similarly, while operations may be depicted in the drawings in a particular order, it is to be recognized that such operations need not be performed in the particular order shown or in sequential order, or that all illustrated operations be performed, to achieve desirable results. Further, the drawings may schematically depict one more example processes in the form of a flowchart. However, other operations that are not depicted may be incorporated in the example methods and processes that are schematically illustrated. For example, one or more additional operations may be performed before, after, simultaneously, or between any of the illustrated operations. Additionally, the operations may be rearranged or reordered in other implementations. In certain circumstances, multitasking and parallel processing may be advantageous. Moreover, the separation of various system components in the implementations described above should not be understood as requiring such separation in all implementations, and it should be understood that the described program components and systems may generally be integrated together in a single software product or packaged into multiple software products. Additionally, other implementations are within the scope of the following claims. In some cases, the actions recited in the claims may be performed in a different order and still achieve desirable results.

Further, while the methods and devices described herein may be susceptible to various modifications and alternative forms, specific examples thereof have been shown in the drawings and are herein described in detail. It should be understood, however, that the invention is not to be limited to the particular forms or methods disclosed, but, to the contrary, the invention is to cover all modifications, equivalents, and alternatives falling within the spirit and scope of the various implementations described and the appended claims. Further, the disclosure herein of any particular feature, aspect, method, property, characteristic, quality, attribute, element, or the like in connection with an implementation or embodiment can be used in all other implementations or embodiments set forth herein. Any methods disclosed herein need not be performed in the order recited. The methods disclosed herein may include certain actions taken by a practitioner; however, the methods can also include any third-party instruction of those actions, either expressly or by implication. The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (e.g., as accurate as reasonably possible under the circumstances, for example ±5%, ±10%, ±15%, etc.). For example, “about 3.5 mm” includes “3.5 mm.” Phrases preceded by a term such as “substantially” include the recited phrase and should be interpreted based on the circumstances (e.g., as much as reasonably possible under the circumstances). For example, “substantially constant” includes “constant.” Unless stated otherwise, all measurements are at standard conditions including temperature and pressure.

As used herein, a phrase referring to “at least one of” a list of items refers to any combination of those items, including single members. As an example, “at least one of: A, B, or C” is intended to cover: A, B, C, A and B, A and C, B and C, and A, B, and C. Conjunctive language such as the phrase “at least one of X, Y and Z,” unless specifically stated otherwise, is otherwise understood with the context as used in general to convey that an item, term, etc. may be at least one of X, Y or Z. Thus, such conjunctive language is not generally intended to imply that certain implementations require at least one of X, at least one of Y, and at least one of Z to each be present. The headings provided herein, if any, are for convenience only and do not necessarily affect the scope or meaning of the devices and methods disclosed herein.

Accordingly, the claims are not intended to be limited to the embodiments or implementations shown herein, but are to be accorded the widest scope consistent with this disclosure, the principles and the novel features disclosed herein.

What is claimed is:

1. A fluid irrigation and/or aspiration apparatus comprising:
a reusable portion comprising:
a pump motor and
a power source; and
a disposable portion comprising:
a handpiece;
a power line connected to the handpiece and connectable to the power source; and
a diaphragm,
wherein, when the reusable portion is connected to the disposable portion at a connection:
the power source interfaces with the power line to deliver power to the handpiece,
the pump motor interfaces with the diaphragm to create a diaphragm pump configured to provide a flow of a fluid in the disposable portion; and
the disposable portion separates the fluid from the reusable portion.

2. The apparatus of claim 1, further wherein the fluid is an irrigation fluid, the apparatus further comprising a source of the irrigation fluid, and wherein the diaphragm pump provides the irrigation fluid to the handpiece.

3. The apparatus of claim 1, further comprising a collector, and wherein the diaphragm pump provides flow of aspirated fluid from the handpiece to the collector.

4. The apparatus of claim 1, wherein the disposable portion further comprises a tube to carry the flow of the fluid.

5. The apparatus of claim 4, further comprising a filter in the tube.

6. The apparatus of claim 5, wherein the filter is located to protect the diaphragm pump from particles in the flow of the fluid.

7. The apparatus of claim 1, wherein the connection comprises a coupling mechanism that transmits motion from the diaphragm pump to the diaphragm.

8. The apparatus of claim 1, wherein the connection comprises one or more sensing devices to sense flow, bubbles, and/or pressure.

9. The apparatus of claim 1, wherein the disposable portion comprises one or more sensing devices to sense flow, bubbles, and/or pressure.

10. The apparatus of claim 1, further comprising a collector wherein the flow is an aspiration from the handpiece directed to the collector.

11. The apparatus of claim 10, wherein the collector is a removable collection bag.

12. The apparatus of claim 1, further comprising a valve.

13. The apparatus of claim 1, wherein the handpiece further comprises identification electronics.

14. A disposable fluid irrigation and aspiration apparatus comprising:
a power line configured to connect to a handpiece and connectable to a power source in a reusable console;
an irrigation diaphragm;
an aspiration diaphragm; and
a connection area configured to connect to the reusable console wherein, when the connection area is connected to the reusable console:
the power line receives power to be delivered to a handpiece,
the irrigation diaphragm interfaces with a pump motor in the reusable console to provide an irrigation flow,
the aspiration diaphragm interfaces with a pump motor in the reusable console to provide an aspiration flow; and
the irrigation flow and the aspiration flow are separated from the reusable console.

15. The apparatus of claim 14, further comprising a filter.

16. The apparatus of claim 14, further comprising the handpiece.

17. The apparatus of claim 14, further comprising one or more sensing devices to sense flow, bubbles, and/or pressure.

18. The apparatus of claim 14, further comprising a collector wherein the aspiration flow directs fluid from the handpiece to the collector.

19. The apparatus of claim 18, wherein the collector is a removable collection bag.
